(19) United States
(12) Patent Application Publication
(10) Pub. No.: US 2014/0216469 A1
(43) Pub. Date: Aug. 7, 2014

(54) MANDIBULAR APPLIANCE WITH OXYGEN

(71) Applicants: Bryan Keropian, Woodland Hills, CA (US); Brian Spriggs, Everett, WA (US)

(72) Inventors: Bryan Keropian, Woodland Hills, CA (US); Brian Spriggs, Everett, WA (US)

(21) Appl. No.: 14/248,182

(22) Filed: Apr. 8, 2014

Related U.S. Application Data
(63) Continuation-in-part of application No. PCT/US13/29067, filed on Mar. 5, 2013.
(60) Provisional application No. 61/607,720, filed on Mar. 7, 2012.

(51) Int. Cl. A61F 5/56; A61M 16/10; A61M 16/04

(52) U.S. Cl. CPC A61F 5/566; A61M 16/0493; A61M 16/1005
USPC 128/847

(57) ABSTRACT

A dental oral appliance to open the airway for a sleeping individual who suffers with snoring or sleep apnea comprising, a mandibular structure configured to removably affix the appliance to the upper and lower teeth, structure attached to said mandibular structure to advance the lower teeth forward, and one or more tubes extending from the anterior of the appliance to the posterior of the appliance, the tubes being connected to a supply of 21% to 100% oxygen.

### CROSS-REFERENCE TO RELATED APPLICATIONS

This application is a continuation-in-part of Patent Application No. PCT/US13/29067, filed Mar. 5, 2013, which claims the benefit of U.S. Provisional Application No. 61/607,720, filed Mar. 7, 2012, the disclosures of which are incorporated herein by reference in their entirety.

### TECHNICAL FIELD

This invention is a dental oral appliance for use by patients who suffer with sleep disorders, to reduce or eliminate snoring and to open the airway for a sleeping individual who suffers with obstructive sleep apnea and to deliver oxygen to the open airway.

### BACKGROUND

It has been estimated that ninety million American adults and children snore and that three in every ten adults snores. Snoring can have serious medical consequences for some people. Snoring is the first indication of a potentially life-threatening sleep disorder called Obstructive Sleep Apnea. If not diagnosed or if left untreated, Obstructive Sleep Apnea could result in severe medical consequences such as systemic high blood pressure, cardiovascular disease and even sudden death.

Obstructive sleep apnea occurs during sleep when the tongue falls and rolls upward and backward, blocking the airway for 10-90 seconds. These events are measured by spending the night sleeping in a center which measures the number of air blockage events per hour. Less than 5 events per hour is normal. 5-19 events per hour is mild sleep apnea. 20-39 events per hour is moderate sleep apnea. Over 40 events per hour is severe sleep apnea.

For sleep apnea there are three main treatments of choice: the CPAP machine, surgery and oral sleep appliances. They are all designed to open the airway during sleep so that there is minimal or no air blockage.

Snoring is caused by vibration of the tissues due to air turbulence as the airway narrows and may be a sign that a patient is suffering from apnea. But not all snorers suffer from apnea. Snoring can be categorized by its severity. There is the snorer who snores but experiences no physical problems. Then, there is the snorer who suffers from apnea, or the snorer who suffers from upper airway resistance. In some of these people, though they may not actually experience 40 apneic episodes, their snoring is so loud and their breathing so labored, that it still wakes them, and their partners, numerous times throughout the night.

Many spouses, partners and/or children suffer through the night from the annoying noise of the snorer. Snoring not only disturbs the sleeping pattern of the snorer himself, it is also disruptive to the family life by causing lack of sleep to all involved. This leaves all involved unrefreshed, tired and sleepy throughout the day. It can cause sleepiness while driving, reading, working or doing other tasks.

A broad variety of intra-oral and dental appliances and devices are now available to treat a patient for snoring. Some known oral devices for treating snoring and obstructive sleep apnea are worn inside of the mouth and work by repositioning of the jaw, moving the mandible forward, lifting the soft palate or moving the tongue forward. These appliances work by advancing the tongue and soft palate away from the back wall of the throat.

Other methods used to treat snoring include controlled positive air-flow pressure systems, also known as CPAP, which require a nose mask and which are quite uncomfortable.

Still other treatments for snoring include various surgeries, which are drastic steps to take to attempt to cure the problem, however snoring can be so disruptive to a person's life and relationships, that some sufferers resort to surgery.

### SUMMARY OF THE INVENTION

The sleep appliance of this invention utilizes mandibular advancers, which move the lower jaw forward to open the oropharyngeal airway. Added to mandibular advancer are one or more tubes that extend from the front, anterior, of the appliance to the back, posterior. The front end of the tubes are connected to an oxygen supply, or a machine that will deliver oxygen through the tubes, into the open oropharyngeal airway. There are many different mandibular advancers now in use, but they all have a method of thrusting the jaw forward.

The mandibular advancer fits over both the upper and lower arch (teeth) as shown in the drawings presented herein.

On the tongue (lingual) side of an upper or lower mandibular advancer is one or more tubes of approximately 0.5 mm, or more, in circumference, extending from about 2 inches to about 12 inches or more, preferably about 6 inches, anterior of the lateral incisors, along either one or both sides of the appliance, to as far back as the posterior extension of the body of the appliance, on both the right and left side. The tubes are for the delivery of oxygen. The tubes can be constructed of any material that is compatible with the environment of the mouth, preferably plastic.

In addition to the basic design of two delivery tubes of oxygen, there can be only one tube, or a plurality of tubes, as many tubes as are required to provide sufficient oxygen for the patient. The addition of feeding oxygen to the sleeping patient, through the sleep appliance worn by the patient, makes his or her sleep more comfortable by reducing or eliminating snoring or sleep apnea.

The tubes for the appliance, for instance, are on the lingual (tongue) side of the appliance, placed anywhere from about 1 mm above the occlusal table, to as high up as the highest extension of the acrylic appliance, as it fits against the palate. The tubes are held in place by any one of a plurality of mechanisms that will keep the tubes attached to the body of the appliance. The tube can be completely or partially encased in plastic as it travels back from the lateral incisors to its most posterior extension, at the posterior of the appliance.

The tubes of the lower appliance are on the lingual (tongue) side of the appliance, placed anywhere from 1 mm below the occlusal table, to as low as the lowest extension of the acrylic, as it fits against the lingual walls of the mandible. The tubes are held in place by any one of a plurality of mechanisms that will keep the tubes attached to the appliance. The tubes can be completely or partially encased in plastic as they travel back from the lateral incisors to their most posterior extension at the posterior of the appliance.

### BRIEF DESCRIPTION OF THE DRAWINGS

FIG. 1A is perspective view of a typical mandibular appliance presently in use.

FIG. 1B is a perspective view of an appliance of this invention, showing air tubes extending from the anterior to the posterior of the appliance.

FIG. 2A is a perspective view of another typical mandibular appliance presently in use.

FIG. 2B is a perspective view showing an appliance of this invention showing air tubes extending into the anterior of the appliance running to the posterior.

FIG. 3 is a top view of the upper portion of an appliance with tubes.

FIG. 4 is a top view of the lower portion of an appliance with tubes.

FIG. 5 is a rear view of either an upper or lower portion of an appliance.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

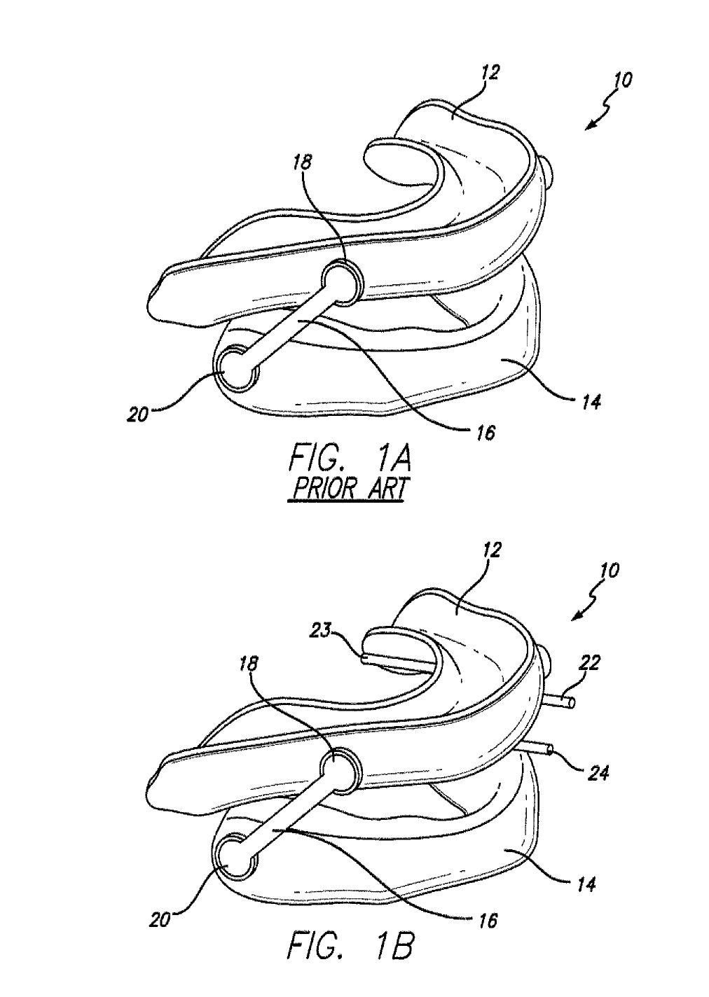

Referring now to the drawings, there is shown in FIG. 1A, a prior art mandibular advance appliance 10. The appliance 10 has an upper occlusal coverage 12 to hold the appliance firmly to the upper teeth. There is also a lower occlusal coverage 14 to hold the appliance firmly to the lower teeth. A firm strap 16, made of plastic, vinyl or other firm material, is attached at the upper end 18 to the upper occlusal coverage 12 and is attached at lower end 20 to the lower occlusal coverage 14. Strap 16 is attached at ends 18 and 20 by a button, snap, clasp or other connector, in order to pull the mandible forward. Strap 16 is attached at an angle so that it can pull the lower jaw forward by forcing the lower occlusal coverage 14 forward, to open the airway. The mandible advancer described above is one of a multitude of designs that fall into the category of Mandibular Devices (Sleep Appliances).

Referring to FIG. 1B, a pair of tubes 22 and 24 are attached to upper occlusal coverage 12 entering at the anterior portion of occlusal coverage 12 and both tubes passing to the posterior 23 as shown, tube 24 also passing to the posterior but not seen in FIG. 1B. A source of oxygen (not seen) would be attached to the anterior of the tubes to carry oxygen into the oropharyngeal airway to help the patient reduce snoring or sleep apnea. The tubes for the passage of oxygen will work with any of the multitude of mandibular devices.

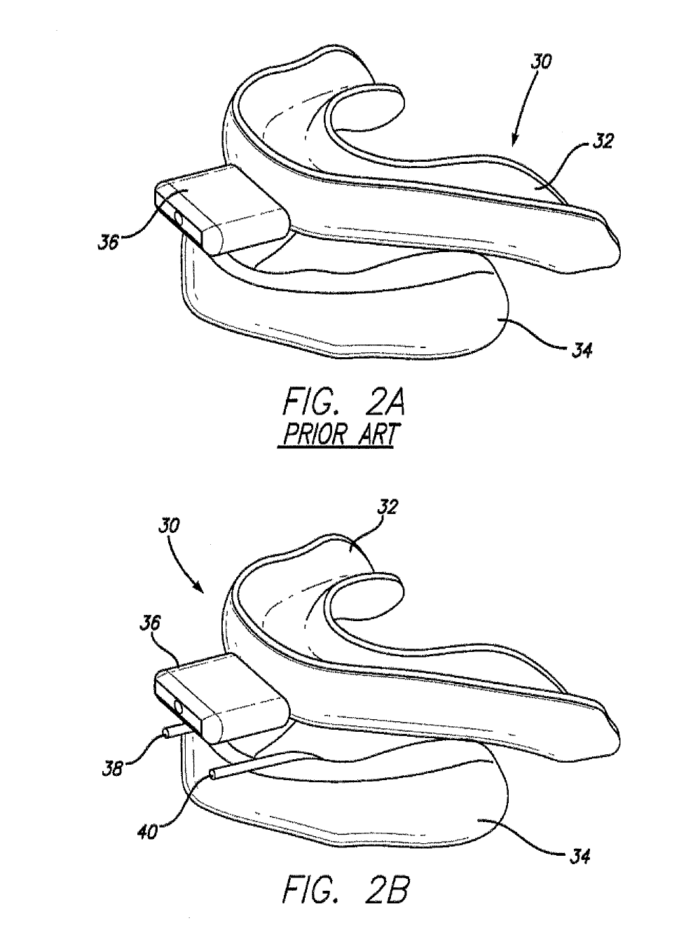

Referring to FIGS. 2A and 2B, the same situation is shown. FIG. 2A shows a prior art mandibular advancer 30, with an upper occlusal coverage 32 and a lower occlusal coverage 34. An attachment device 36 is attached to lower occlusal coverage 34 and can be turned to cause the lower jaw to be advanced. Attachment 36 is rotated or manipulated to push the jaw forward (in the lower occlusal coverage 34) to open the oropharyngeal airway. Oxygen tubes 38 and 40 are attached to the anterior portion of lower occlusal coverage 34 and pass to the posterior area to deliver the oxygen.

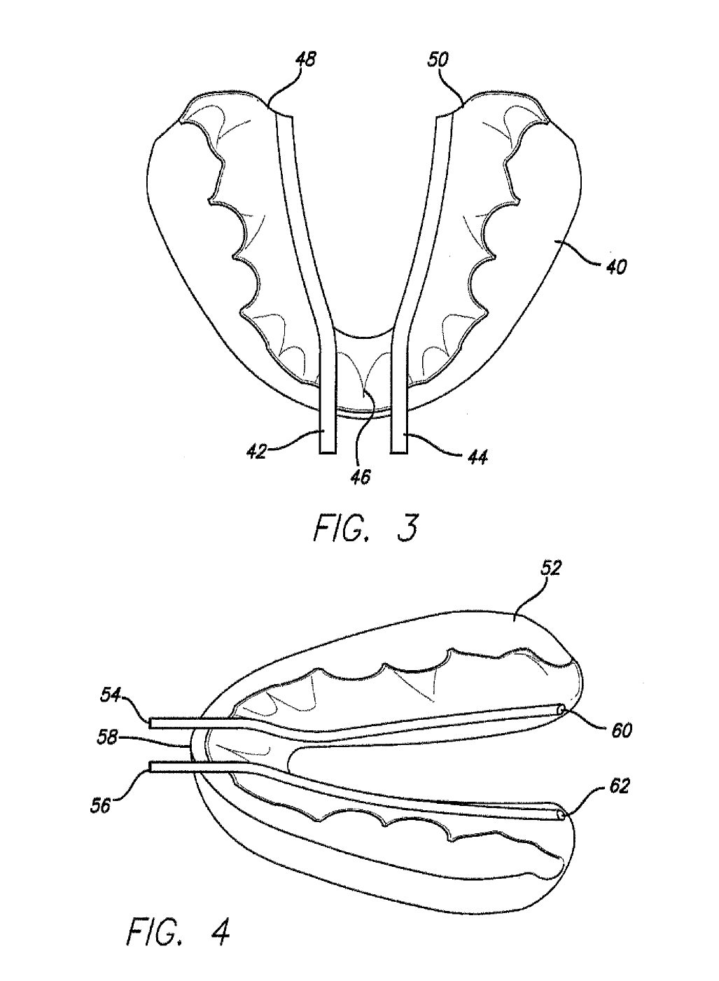

Referring to FIG. 3, there is shown the view from the upper biting surface, an upper occlusal coverage 40 with oxygen tubes 42 and 44. Tubes 42 and 44 run from the anterior portion 46 of occlusal coverage 40 to the posterior portion 48 and 50 to carry oxygen to the posterior portion into the oropharyngeal airway.

Referring to FIG. 4, there is shown the view from a lower biting surface, a lower occlusal coverage 52 with oxygen tubes 54 and 56. Tubes 54 and 56 run from the anterior portion 58 of occlusal coverage 52 to the posterior portion 60 and 62 to carry oxygen to the posterior portion into the oropharyngeal airway.

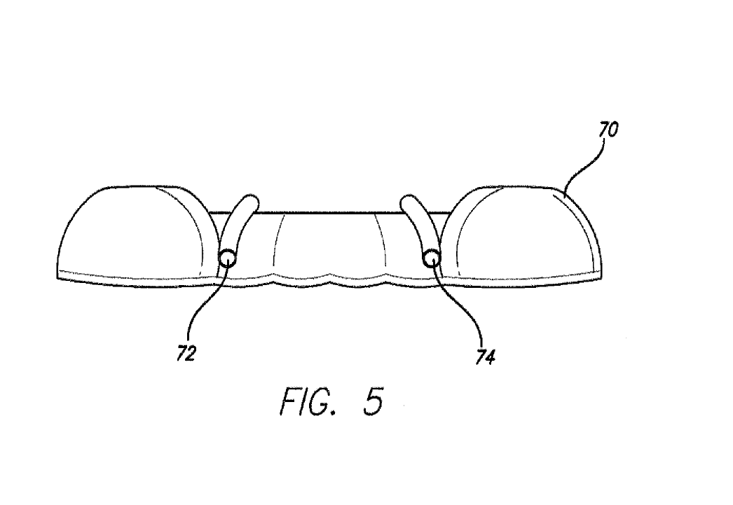

Referring to FIG. 5, there is shown an upper or a lower view from the posterior of an occlusal coverage 70. Oxygen tubes 72 and 74 are attached to occlusal coverage 70.

The occlusal coverage of each appliance shown comprises a body which fits adjacent the inside of the upper or lower teeth of the person wearing it. The body is generally U-shaped with an open palate and is custom fitted to fit over the wearer's upper or lower teeth, in the same manner as an occlusal night guard, which uses an occlusal coverage. The occlusal coverage holds appliance firmly onto the patient's teeth. The appliance is made using a clear acrylic plastic commonly used for dental appliances.

The oxygen tubes shown in the drawings are on the lingual (tongue) side of either an upper or lower sleep appliance. The tubes have a circumference of about 0.5 mm, or more, and extend anterior of the lateral incisors, along either or both sides of the appliance, to as far back as the posterior extension of the body of the appliance, on both the right and left sides. The tubes are for the delivery of oxygen or air or a mixture thereof. The tubes can be constructed of any material that is compatible with the environment of the mouth, preferably plastic.

The tubes can be fastened to the body of the appliance by the use of acrylic plastic covering some or all of the tubes. The tubes can extend as far as 12 inches or more anterior of the lateral incisors, and will be connected to the supply of oxygen or air which will be fed through the tubes to the wearer's oropharyngeal airway.

While two air tubes are shown in the drawings described, there may be only one tube or a plurality of tubes connected to the appliance, depending upon the needs of the patient. The tubes described herein are placed for the delivery of oxygen from an oxygen canister, an oxygen concentrator or any device which delivers oxygen from 21% to 100% oxygen.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A dental oral appliance to open the airway for a sleeping individual who suffers with snoring or sleep apnea comprising, a mandibular structure configured to removably affix the appliance to the upper and lower teeth, structure attached to said mandibular structure to advance the lower teeth forward, and one or more tubes extending from the anterior of the appliance to the posterior of the appliance.

2. The dental oral appliance of claim 1 in which the structure to removably affix the appliance to the upper and lower teeth is an upper occlusal coverage and a lower occlusal coverage.

3. The dental oral appliance of claim 1 in which advancing the lower teeth forward opens the oropharyngeal airway.

4. The dental oral appliance of claim 1 in which the mandibular structure has an anterior portion and a posterior portion.

5. The dental oral appliance of claim 4 in which the tubes are attached to the anterior portion and posterior portion of the mandibular structure.

6. The dental oral appliance of claim 5 in which the tubes are attached to the mandibular structure by acrylic plastic.

7. The dental oral appliance of claim 1 in which the tubes are attached to the entire length of the mandibular structure.

8. The dental oral appliance of claim 1 in which a source of oxygen is attached to the anterior end of the tubes.

9. The dental oral appliance of claim 2 in which the tubes are attached to the upper occlusal coverage or the lower occlusal coverage.

10. The dental oral appliance of claim 1 in which the tubes are attached to a supply of oxygen.

11. A dental oral appliance to open the oropharyngeal airway for a sleeping individual who suffers with snoring or sleep apnea comprising, a mandibular structure having an upper occlusal coverage and a lower occlusal coverage removably affixed to the upper and lower teeth, structure attached to said mandibular structure to advance the lower teeth forward, and one or more tubes extending from the front to the back of the appliance, the tubes being attached to a supply of oxygen, air or a mixture thereof.

12. The dental oral appliance of claim 11 in which the tubes are attached to the mandibular structure by acrylic plastic.

13. The dental oral appliance of claim 11 in which the tubes are attached to the entire length of the mandibular structure.

14. The dental oral appliance of claim 11 in which the tubes are attached to the upper occlusal coverage or the lower occlusal coverage.
